(19) United States
(12) Patent Application Publication
(10) Pub. No.: US 2026/0013727 A1
(43) Pub. Date: Jan. 15, 2026

(54) SYMBIOTIC WEARABLE PLATFORM FOR HEALTH MONITORING AND INTERVENTION

(71) Applicant: Abdallah Zeyad Almajdoub, Makkah (SA)

(72) Inventor: Abdallah Zeyad Almajdoub, Makkah (SA)

(21) Appl. No.: 19/334,876

(22) Filed: Sep. 21, 2025

(51) Int. Cl. A61B 5/00; A61B 5/024; A61B 5/103; A61B 5/11; H04L 9/40

(52) U.S. Cl. CPC A61B 5/0015; A61B 5/02427; A61B 5/1038; A61B 5/112; A61B 5/4836; A61B 5/6807; A61B 5/7267; H04L 63/0861; A61B 2560/0209; A61B 2562/0247

(57) ABSTRACT

A symbiotic wearable electronic platform (100) is disclosed, adaptable to various form factors. It features a processing unit (102), a rechargeable battery (410), and a synergistic multi-source power system (400) integrating at least two energy harvesting modalities solar (402), kinetic (404), and wireless (406) for near-perpetual operation. A multi-modal sensor array (500) synergistically fuses data from biomechanical (502), physiological (506), and biochemical (508) sensors. A hardware-secured biometric authentication system utilizes a Trusted Execution Environment (TEE). A closed-loop therapeutic system with EAP actuators (1402) provides real-time intervention. The platform also enhances product sustainability with a modular design and material degradation sensing. This invention provides a holistic technical solution to manifold limitations of the prior art. The inventive concepts are claimed individually and collectively.

### CROSS-REFERENCE TO RELATED APPLICATIONS

This application does not claim the benefit of any earlier-filed provisional or non-provisional application, nor is it a continuation, continuation-in-part, or divisional application of any earlier-filed application.

### BACKGROUND OF THE INVENTION

### 1. Field of the Invention

The present invention relates generally to the field of wearable electronic devices, remote patient monitoring (telemedicine), and human-machine interfaces. More specifically, the invention pertains to a symbiotic, self-sustaining wearable platform for health monitoring that incorporates a distributed, multi-modal sensor array, a synergistic energy harvesting and management system, and, in certain embodiments, a closed-loop interactive feedback system for active therapeutic intervention. The strategic value of this invention lies in its architectural framework as a “platform” rather than a single-purpose device. This approach aligns with the patent strategies of market leaders, which focus on securing broad, foundational technologies that can support a wide range of products and an interconnected ecosystem of devices and services. This invention follows a similar philosophy, protecting the core technological pillars that enable a versatile, scalable, and defensible product line, thus creating a significant competitive advantage.

### 2. Description of Related Art

Despite significant advancements, the existing art in wearable technology suffers from a cascade of critical and interconnected technical deficiencies that collectively hinder its utility for continuous, long-term, and clinical-grade applications. This “cascade of limitations” represents a long-felt but unsolved need in the technical field for a truly integrated and autonomous wearable health solution, which the present invention directly addresses.

The present invention is a direct technical solution to each of these problems, distinguishing it from mere incremental improvements. This TABLE 1 demonstrates how each inventive concept provides a direct technical solution to a specific problem identified in the prior art, distinguishing the invention from mere incremental improvements.

The detailed technical and reference data are provided in the following tables: TABLE 2 provides a comprehensive master list of all reference numbers and their descriptions. TABLE 3 offers a detailed bill of materials for the Main Hub. TABLE 4 outlines the inventive solutions and their corresponding technical components. TABLE 5 presents the technical specifications of the tri-hybrid power system, and TABLE 6 details the specifications of the key sensors, including their synergistic value.
a. The Problem of Power Dependency & Data Gaps: The technical community has been grappling with this fundamental power problem for decades, and until now, the prior art has not produced a robust, self-sustaining solution. The overwhelming majority of existing wearable devices rely on a single, internal battery that requires frequent, often daily, recharge. This fundamental operational constraint leads to significant and unavoidable gaps in data collection, rendering these devices impractical for applications demanding uninterrupted, 24/7 monitoring, such as clinical trials or the management of chronic conditions. While prior art patents such as U.S. Pat. No. 10,663,925B2 and U.S. Pat. No. 20,160,261031A1 disclose combining multiple energy sources, they do not disclose a system that uses these sources in a synergistic, “tri-hybrid” manner with a sophisticated management circuit specifically designed for “near-perpetual operation” to eliminate data gaps for continuous clinical-grade monitoring. Their focus is merely on extending the operating time of the device, not on enabling the continuous, rich data stream required for advanced applications.
b. The Problem of a Constrained Sensory Scope: The technical limitations imposed by power-constrained designs have long prevented the holistic integration of diverse sensor modalities, problem experts in the field have been unable to overcome. As a direct consequence of power limitations, the prior art has focused on integrating low-power, uni-modal sensors, such as basic accelerometers and pressure sensors. There is a conspicuous lack of systems that holistically and synergistically integrate these with more power-intensive modalities, such as physiological and biochemical sensors, to provide a richer, multi-faceted view of a user's health state. For example, the patent application U.S. Pat. No. 20,180,160966A1 describes a multi-modal system for joint health, but it is limited to a specific application and does not teach the broad, synergistic fusion of biomechanical, physiological, and biochemical data to generate a general composite health metric. This invention overcomes these limitations through a symbiotic approach that directly links a self-sustaining power system to an expanded sensory scope, ensuring that sensory capabilities are never constrained by power limitations.
c. The Problem of a Passive Monitoring Paradigm: Despite the clear desire for interactive therapeutic devices, the technical challenge of powering active components has resulted in a pervasive ‘passive monitoring’ paradigm that has frustrated inventors for years. Prior art devices are primarily designed for passive data collection and simple alerts. They fundamentally lack the technical capability for integrated, closed-loop, and active therapeutic intervention due to the significant power required to operate active components like actuators. While some smart insoles, such as those described in WO2009089406A2, utilize pressure sensors, they do not teach the use of active EAP actuators for dynamic, closed-loop gait correction or therapeutic intervention.
d. The Problem of a Long-standing Security Vulnerability: The challenge of securing sensitive biometric data on low-power wearable devices has been a well-known problem, with a clear and long-felt need for a robust, hardware-based security solution that the prior art has not yet addressed with the present solution. With the increasing collection of sensitive Personal Health Information (PHI), security is a paramount concern. Biometric identifiers are often processed at the software level, which exposes this sensitive data to a wide range of software-based attacks. For example, patent WO2016105892A1 discloses a method for authentication using a user's body chemistry, but it does not mention the use of a hardware-isolated trusted execution environment (TEE) to perform the entire authentication process securely, which is the core innovation of the present invention.
e. The Problem of a Pervasive Sustainability Crisis: The societal problem of electronic waste has existed for decades, yet a viable technical solution for creating long-lasting, repairable wearable devices remained elusive. The consumer electronics industry is characterized by short product lifecycles and the generation of substantial electronic waste (e-waste). The prior art offers no integrated solution to extend product longevity and reduce environmental impact.

TABLE 1
MAPPING PRIOR ART LIMITATIONS TO INVENTIVE SOLUTIONS

| | Inventive Technical Solution Provided |
|---|---|
| Prior Art Limitation | by the Present Invention |
| 1. Power Dependency & Data Gaps | Synergistic Tri-Hybrid Power System (400) for near- |
| | perpetual operation. |
| 2. Limited, Uni-modal Sensing | Distributed Multi-Modal Sensor Array (500) with |
| | Synergistic Fusion logic. |
| 3. Passive Monitoring Paradigm | Active Therapeutic System with Electroactive Polymer |
| | (EAP) Actuators (1402) for closed-loop intervention. |
| 4. Software-Level Security Risks | Hardware-Secured Biometric Authentication via a |
| | Trusted Execution Environment (TEE). |
| 5. Short Lifecycles & E-Waste | Modular Architecture and a Method for Material |
| | Degradation Sensing. |

### BRIEF SUMMARY OF THE INVENTION

In accordance with various embodiments of the present disclosure, a wearable electronic platform is provided that overcomes the manifold limitations of the prior art. The present invention is architected as a symbiotic platform wherein each innovative pillar provides a direct, non-obvious technical solution to a fundamental problem that has long plagued the technical community, thereby collectively distinguishing the invention from mere incremental improvements in the prior art.

In some embodiments, a device comprises a processing unit (102), an internal rechargeable battery (410), a multi-source power system (400), and a multi-modal sensor array (500). The multi-source power system (400) is operatively connected to the processing unit and the battery, and includes at least two distinct energy harvesting modalities selected from a solar circuit (402), a kinetic circuit (404), and a wireless charging circuit (406). This system intelligently manages energy flow to provide a technical solution to the foundational power dependency problem, enabling continuous operation and a rich data stream.

The multi-modal sensor array (500) is configured to synergistically fuse data from at least one biomechanical sensor (502), one physiological sensor (506), and one biochemical sensor (508). The processing unit (102) is configured to execute a synergistic fusion algorithm that combines these disparate data streams to generate composite health metrics, providing a more comprehensive and actionable understanding of a user's health state.

The relationship between these technical pillars is not one of simple aggregation, but a truly symbiotic one. The synergistic tri-hybrid power system (400) is not merely designed to extend battery life; it is the fundamental enabler that ensures a continuous, rich data stream from the multi-modal sensor array (500). This intimate interdependence between the power source and the data aggregator is a cornerstone of the platform's design, ensuring that each system supports and enhances the function of the other, thereby fundamentally solving the prior art's challenges of data gaps and constrained sensory scope.

Furthermore, the processing unit (102) includes a hardware-isolated Trusted Execution Environment (TEE) for a novel biometric authentication method. A user's unique biometric signature is generated, stored, and verified entirely within the secure, hardware-enforced confines of the TEE, thus providing a high level of security against software-based attacks.

In certain embodiments, such as an article of footwear, the platform includes an active, closed-loop therapeutic system that uses an array of Electroactive Polymer (EAP) actuators (1402). This enables the device to move beyond passive monitoring to provide active, real-time therapeutic intervention.

### BRIEF DESCRIPTION OF THE SEVERAL VIEWS OF THE DRAWING

The accompanying drawings, which are incorporated in the specification, illustrate various embodiments of the invention and serve to explain its principles in conjunction with the detailed description.

FIG. 01: Is a schematic block diagram illustrating an exemplary architecture of a symbiotic wearable platform.

FIG. 02: Is a cutaway perspective view of an article of footwear embodying the symbiotic wearable platform. The drawing illustrates the position of the Main Hub (212), the Instep & Sides Unit (214), and the Rear Collar Unit (216).

FIG. 03: Is a top-down plan view of an exemplary main rigid printed circuit board (PCB).

FIG. 04: Is a schematic block diagram of an exemplary synergistic, multi-source energy harvesting and power management system.

FIG. 05: Is a schematic block diagram of an exemplary multi-modal sensor array.

FIG. 06: Is a schematic block diagram of an exemplary interactive user feedback system.

FIG. 07: Is a detailed view illustrating an embodiment incorporating a fiber-optic textile.

FIG. 08: Is a schematic diagram illustrating the layered construction of capacitive sensor electrodes for pressure mapping.

FIG. 09: Is a perspective view of an exemplary modular smart insole.

FIG. 10: Is a system-level block diagram of the electronic architecture.

FIG. 11: Is a simplified circuit schematic of an exemplary multi-source power management system.

FIG. 12: Is a flowchart illustrating the steps of an exemplary sensor fusion algorithm for health risk assessment.

FIG. 13: Is a flowchart illustrating the process steps of an exemplary hardware-secured biometric authentication method.

FIG. 14: Is a cross-sectional view of an embodiment featuring an active orthotic layer, shown in a resting state (FIG. 14A) and an actuated state (FIG. 14B).

FIG. 15: Is a perspective view illustrating an exemplary modular upper assembly for a footwear embodiment.

FIG. 16: Is a detailed circuit diagram illustrating the Alternating Actuation Mode for EAP actuators.

FIG. 17: Is a flowchart illustrating the process steps of the method for enhancing product lifecycle sustainability.

FIG. 18: Main Hub Board—A schematic diagram of the Central Processing Unit (U_MCU1).

FIG. 19: Main Hub Board—A schematic diagram of the Tri-Hybrid Energy Harvesting and Power Management Circuit.

FIG. 20: Main Hub Board—A schematic diagram of the Buck-Boost Converter and Auxiliary Power Regulation Circuits.

FIG. 21: Main Hub Board—A schematic diagram of the GNSS Module and I2C Multiplexer.

FIG. 22: Main Hub Board—A schematic diagram of the Capacitive Sensing and Inertial Measurement Unit (IMU) Circuits.

FIG. 23: Main Hub Board—A schematic diagram of the Solar Harvesting and User Interface Circuits.

FIG. 24: Main Hub Board—A schematic diagram of the Ambient Light Sensor, TVS Diodes, and Power/Reset Buttons.

FIG. 25: Instep & Sides Unit—A schematic diagram of the Capacitive Sensing, PPG Sensor, and Haptic Feedback Circuits.

FIG. 26: Instep & Sides Unit—A detailed schematic of the Wireless Charging Circuit.

FIG. 27: Rear Collar Unit—A schematic diagram of the Environmental, PPG, and Haptic Feedback Circuits.

FIG. 28: A detailed layout of the main rigid PCB, showing the strategic placement of the core processing unit, energy harvesting circuits, and key sensors.

FIG. 29: A detailed layout of the flexible PCB for the instep and sides unit, showing the placement of the wireless charging receiver, PPG sensor, and haptic driver.

FIG. 30: A detailed layout of the flexible PCB for the rear collar unit, showing the placement of the environmental sensor, PPG sensor, and haptic driver.

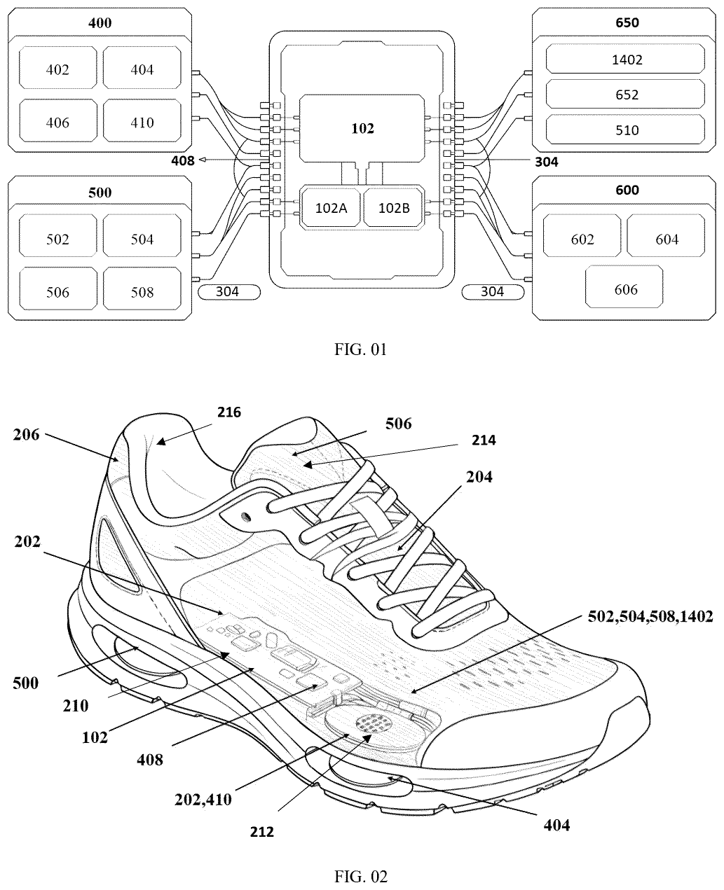

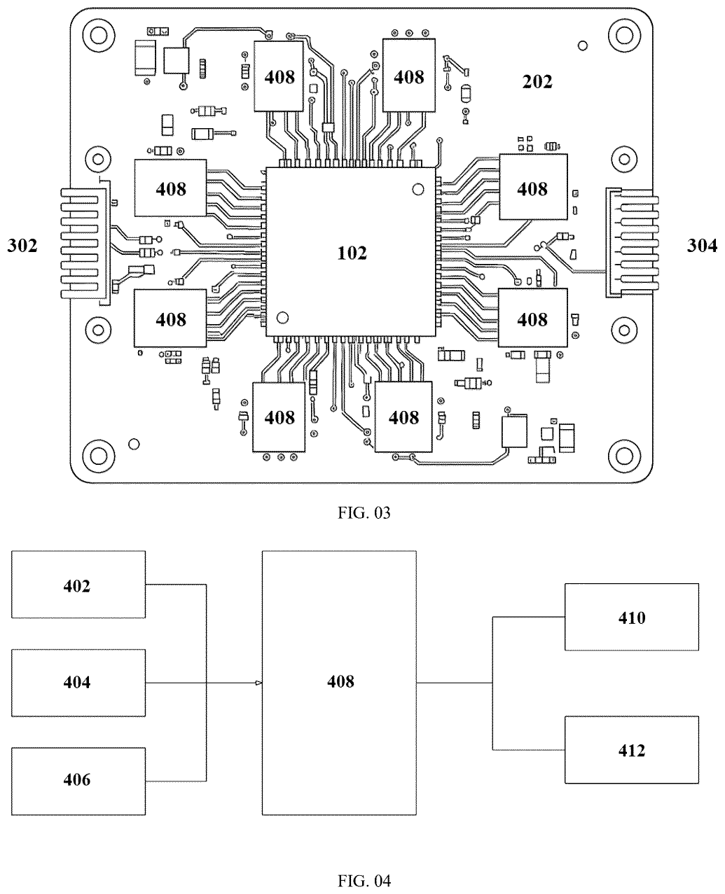

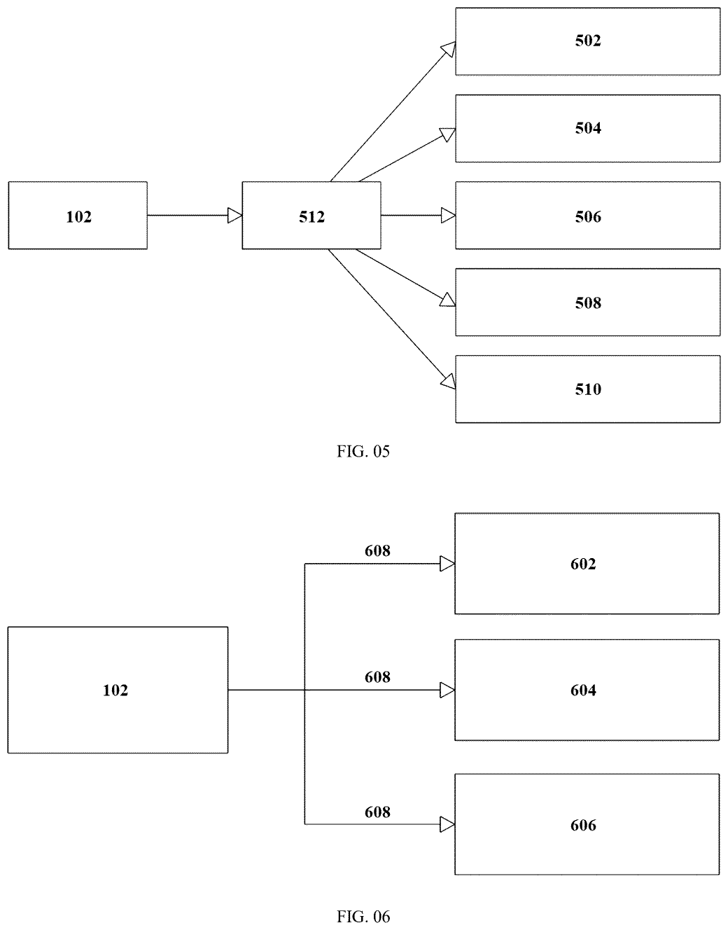

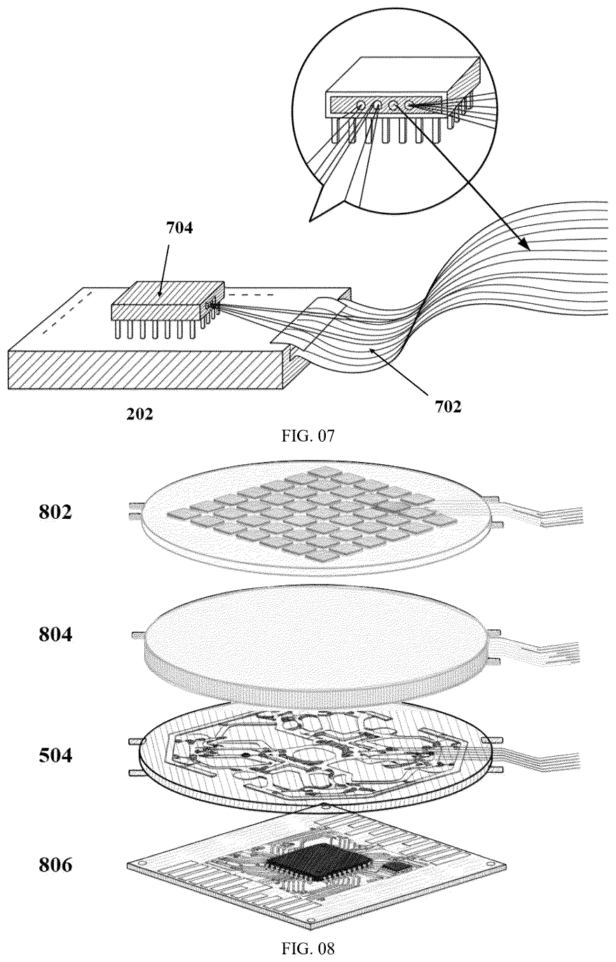

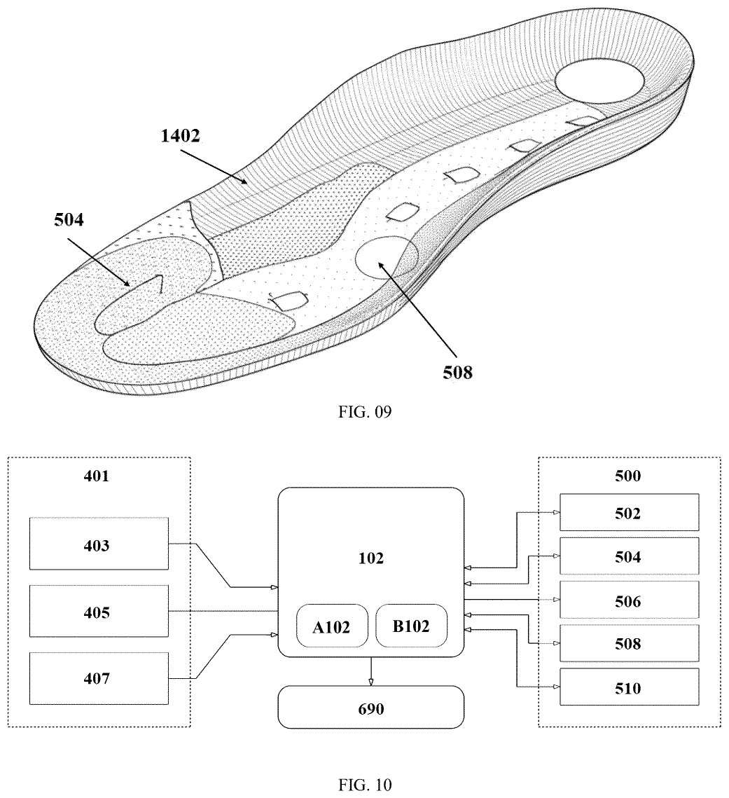

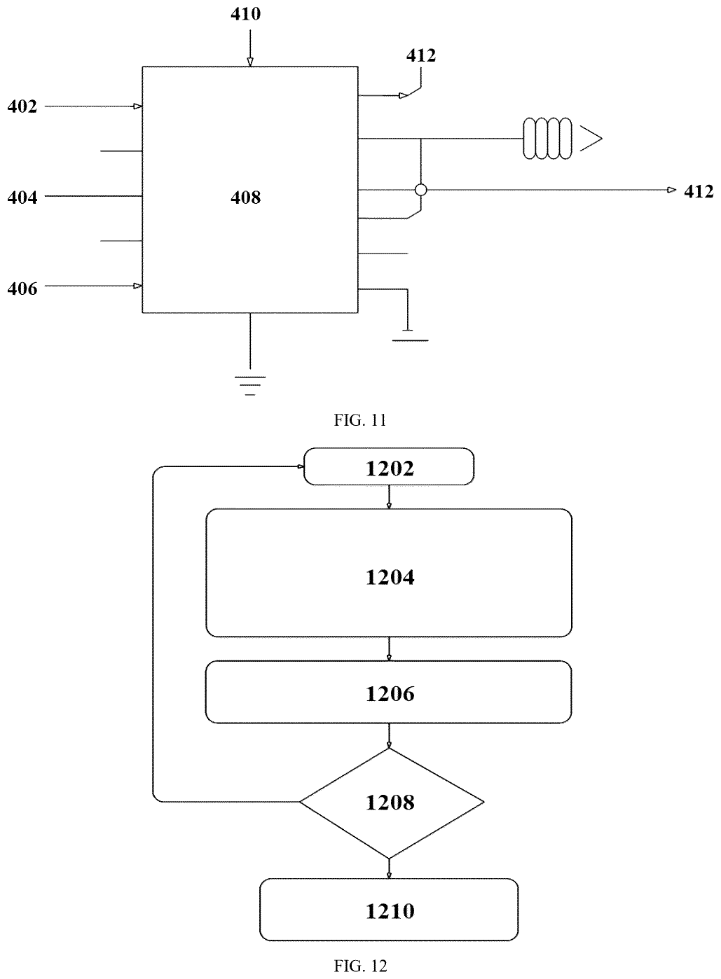

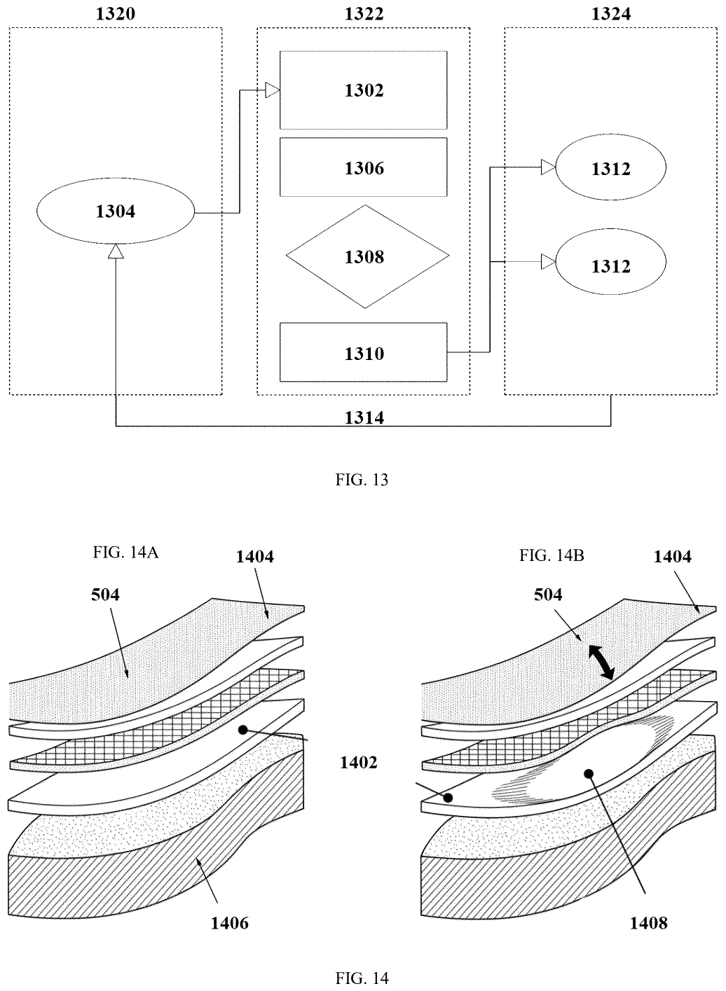

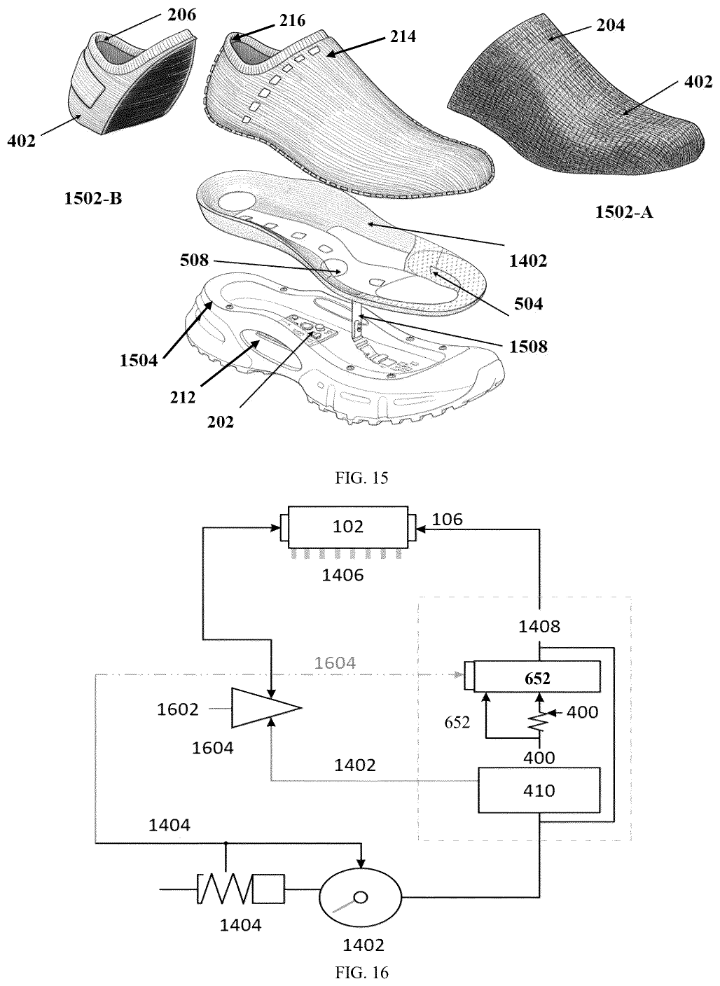

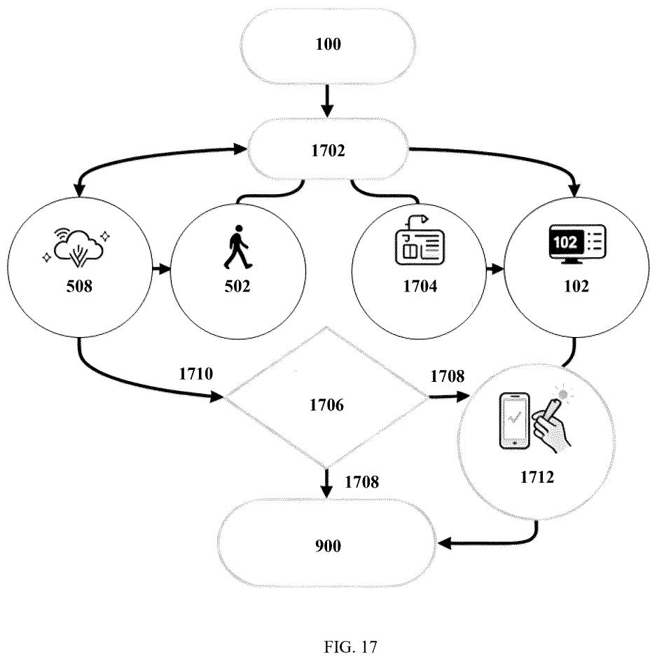

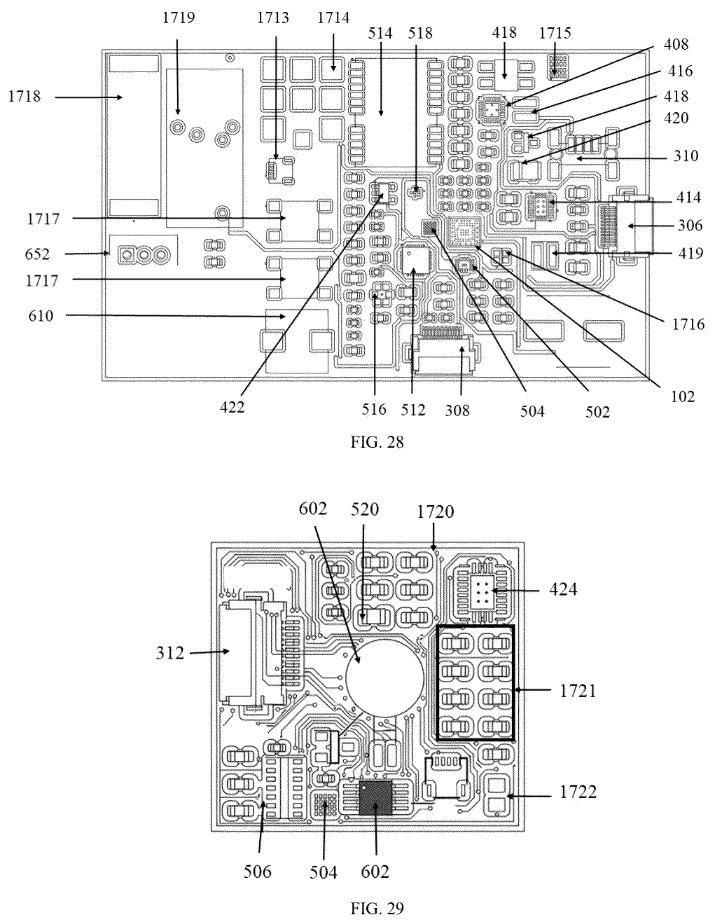

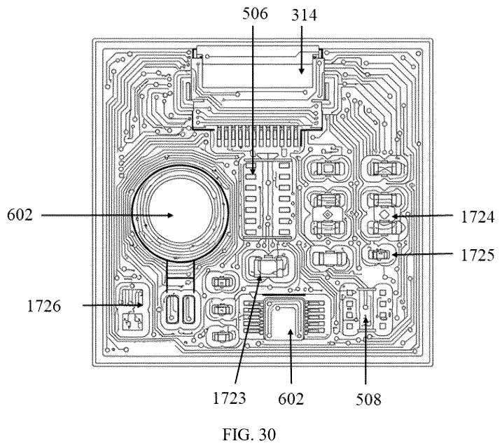

TABLE 2
CONSOLIDATED MASTER REFERENCE TABLE

| Reference | | |
|---|---|---|
| Number | Description | Figure(s) |
| 100 | Footwear embodiment of the symbiotic | FIG. 01, FIG. 02, FIG. 17 |
| | wearable platform | |
| 102 | Processing unit, System-on-Chip (SoC) | FIG. 01, FIG. 02, FIG. 03, FIG. |
| | | 05, FIG. 06, FIG. 10, FIG. 16, |
| | | FIG. 17 |
| A102 | High-performance application core | FIG. 01 |
| B102 | Low-power network core | FIG. 01 |
| 106 | Driver circuit for EAP actuators | FIG. 16 |
| 202 | Main rigid printed circuit board (PCB) | FIG. 02, FIG. 03, FIG. 07, FIG. 28 |
| 204 | Dorsal board, flexible PCB | FIG. 02, FIG. 15, FIG. 29 |
| 206 | Collar board, flexible PCB | FIG. 02, FIG. 15, FIG. 30 |
| 210 | Arch of the shoe's midsole | FIG. 02 |
| 212 | The Main Hub | FIG. 02, FIG. 15 |
| 214 | The Instep & Sides Unit | FIG. 02, FIG. 15 |
| 216 | The Rear Collar Unit | FIG. 02, FIG. 15 |
| 302 | First high-density connector, serving as the | FIG. 03 |
| | physical interface for the Sensor Data Bus | |
| 304 | Second high-density connector, serving as | FIG. 01, FIG. 03 |
| | the physical interface for the System | |
| | Control and Feedback Control Busses | |
| 306 | 12-pin FFC/FPC Connector | FIG. 28 |
| 308 | 12-pin FFC/FPC Connector | FIG. 28 |
| 310 | SWD Programming Connector (M55- | FIG. 28 |
| | 3000442R) | |
| 312 | 12-pin FFC/FPC Connector | FIG. 29 |
| 314 | 12-pin FFC/FPC Connector | FIG. 30 |
| 400 | Multi-source power system, synergistic tri- | FIG. 01, FIG. 16 |
| | hybrid power system | |
| 401 | Power Management System | FIG. 10 |
| 402 | Solar circuit, energy harvesting circuit | FIG. 01, FIG. 04, FIG. 11, FIG. 15 |
| 403 | Energy Harvesting Management | FIG. 10 |
| 404 | Kinetic circuit, energy harvesting circuit | FIG. 01, FIG. 02, FIG. 04, FIG. 11 |
| 405 | Wireless Power Receiver | FIG. 10 |
| 406 | Wireless charging circuit, energy harvesting | FIG. 01, FIG. 04, FIG. 11 |
| | circuit | |
| 407 | Voltage Regulation (Buck-Boost) | FIG. 10 |
| 408 | TI BQ25570RGRR, ultra-low power boost | FIG. 01, FIG. 02, FIG. 03, FIG. |
| | converter, Nano-Power Management PMIC | 04, FIG. 11 |
| | IC | |
| 410 | Internal rechargeable battery | FIG. 01, FIG. 02, FIG. 04, FIG. |
| | | 11, FIG. 16 |
| 412 | System Load for FIG. 4, System Load and | FIG. 04, FIG. 11 |
| | Output for FIG. 11 | |
| 414 | Buck-Boost Converter (TI TPS63020DSJT) | FIG. 28 |
| 416 | Inductor for Buck circuit (L_BUCK1) | FIG. 28 |
| 418 | Diode for energy harvesting circuit | FIG. 28 |
| | (D_HARVEST1) | |
| 419 | Inductor for Buck-Boost converter | FIG. 28 |
| | (L_BUCKBOOST1) | |
| 420 | Inductor for energy harvesting circuit | FIG. 28 |
| | (L_HARVEST1) | |
| 422 | LDO Regulator (AP2112K-1.8TRG1) | FIG. 28 |
| 424 | Wireless Power Receiver IC (TI | FIG. 29 |
| | BQ51013BRHLR) | |
| 426 | 4-Pin FFC/FPC Connector for Wireless Coil | FIG. 29 |
| | (CONN_COIL1) | |
| 500 | Multi-modal sensor array | FIG. 01, FIG. 02, FIG. 10 |
| 502 | Biomechanical Sensor (IMU) | FIG. 01, FIG. 02, FIG. 05, FIG. |
| | | 10, FIG. 17, FIG. 28 |
| 504 | Plantar pressure sensing array (capacitive- | FIG. 01, FIG. 02, FIG. 05, FIG. |
| | to-digital converters) | 08, FIG. 09, FIG. 10, FIG. 14A, |
| | | FIG. 14B, FIG. 15, FIG. 28, FIG. |
| | | 29 |
| 506 | Physiological sensor (PPG) | FIG. 01, FIG. 02, FIG. 05, FIG. |
| | | 10, FIG. 29, FIG. 30 |
| 508 | Biochemical sensor (gas) | FIG. 01, FIG. 02, FIG. 05, FIG. |
| | | 09, FIG. 10, FIG. 15, FIG. 17 |
| 510 | Global Navigation Satellite System (GNSS) | FIG. 01, FIG. 05, FIG. 10 |
| | Unit | |
| 512 | I2C Bus Multiplexer | FIG. 05, FIG. 28 |
| 514 | GNSS Module (u-blox NEO-M9N-00B) | FIG. 28 |
| 516 | Ambient Light Sensor (VEML603 1X00) | FIG. 28 |
| 518 | GNSS Antenna Filter (AFS14A04-1575,42- | FIG. 28 |
| | T3) | |
| 520 | Capacitor for PPG sensor circuit | FIG. 29 |
| | (C_PPG_BULK1) | |
| 522 | Environmental Sensor (Bosch BME688) | FIG. 30 |
| 600 | Interactive User Feedback System | FIG. 01 |
| 602 | Haptic Feedback | FIG. 01, FIG. 06 |
| 604 | Visual Feedback | FIG. 01, FIG. 06 |
| 606 | Acoustic Feedback | FIG. 01, FIG. 06 |
| 608 | Control Signals | FIG. 06 |
| 610 | Magnetic Buzzer (CMI-9651S-SMT-TR) | FIG. 28 |
| 612 | Vibration Motor (C0720B001F) | FIG. 29, FIG. 30 |
| 614 | Haptic Driver IC (TI DRV2605LDGS) | FIG. 29 |
| 616 | Haptic Driver IC (TI DRV2605LDGS) | FIG. 30 |
| 650 | Therapeutic & Navigation Systems | FIG. 01 |
| 652 | High-Voltage Driver (MOSFET Q1) | FIG. 01, FIG. 16, FIG. 28 |
| 690 | Actuator & Feedback System | FIG. 10 |
| 702 | Fiber-optic textile, for advanced sensing or | FIG. 07 |
| | data transmission | |
| 704 | Electro-optical interface, which converts | FIG. 07 |
| | optical signals to electrical signals for the | |
| | PCB | |
| 802 | First layer of electrodes in the capacitive | FIG. 08 |
| | sensor array | |
| 804 | Compressible dielectric layer disposed | FIG. 08 |
| | between the electrode layers | |
| 806 | Flexible substrate upon which the sensor | FIG. 08 |
| | layers are constructed | |
| 900 | Generate User Alert | FIG. 17 |
| 1202 | Acquire Data from Biomechanical Sensors, | FIG. 12 |
| | Start Monitoring | |
| 1204 | Acquire Synchronized Data Streams from | FIG. 12 |
| | Biochemical Sensors | |
| 1206 | Calculate Composite Risk Score | FIG. 12 |
| 1208 | Is Risk Score > Threshold? | FIG. 12 |
| 1210 | Trigger High-Priority Alert to User & | FIG. 12 |
| | Clinician | |
| 1302 | Collect real-time gait data (from IMU 502 | FIG. 13 |
| | & CDCs 504) | |
| 1304 | Receive Authentication Request | FIG. 13 |
| 1306 | Load Secure Template from Memory | FIG. 13 |
| 1308 | Compare Live Data with Secure Template | FIG. 13 |
| 1310 | Is Match Score > Threshold? | FIG. 13 |
| 1312 | Generate Authentication Result | FIG. 13 |
| 1314 | Return Result to Normal World | FIG. 13 |
| 1320 | Normal World | FIG. 13 |
| 1322 | Secure World (TEE) | FIG. 13 |
| 1324 | Authentication | FIG. 13 |
| 1402 | Electroactive Polymer (EAP) actuators | FIG. 01, FIG. 02, FIG. 09, FIG. |
| | | 14A, FIG. 14B, FIG. 16 |
| 1404 | Top fabric layer of the insole, for user | FIG. 14A, FIG. 14B, FIG. 16 |
| | contact and comfort | |
| 1406 | High-Voltage Driver Circuit | FIG. 14A, FIG. 16 |
| 1408 | High-Voltage Boost Converter | FIG. 14B, FIG. 16 |
| 1410 | Capacitance Measurement Circuit | FIG. 16 |
| 1502-A | Detachable section of the modular front | FIG. 15 |
| | assembly | |
| 1502-B | Detachable section of the modular rear | FIG. 15 |
| | assembly | |
| 1504 | Quick-release mechanism (e.g., mechanical | FIG. 15 |
| | snap or magnetic connector) | |
| 1508 | Electrical connector | FIG. 15 |
| 1602 | High-voltage pulse | FIG. 16 |
| 1604 | PWM Signal (Pulse-width modulated | FIG. 16 |
| | signal) | |
| 1702 | Collect Data | FIG. 17 |
| 1704 | Correlate & Analyze Data | FIG. 17 |
| 1706 | Is Component Degradation Detected? | FIG. 17 |
| 1708 | Yes | FIG. 17 |
| 1710 | No | FIG. 17 |
| 1712 | Replace modular component | FIG. 17 |
| 1713 | Piezoelectric sensor connector | FIG. 28 |
| | (CONN_PIEZO1) | |
| 1714 | Component part number (SGGP.4.A) | FIG. 28 |
| 1715 | TVS Diode Array | FIG. 28 |
| | (TVS_DIODE_ARRAY_1,2) | |
| 1716 | Crystal Oscillator (ECS-320-8-37B-CWY- | FIG. 28 |
| | TR) | |
| 1717 | Capacitor (CS11028.0F160) | FIG. 28 |
| 1718 | Component part number (KXXOB25- | FIG. 28 |
| | 05X3F-TR) | |
| 1719 | Component part number (A10P-5) | FIG. 28 |
| 1720 | Resistor for wireless charging circuit | FIG. 29 |
| | (R_ILIM) | |
| 1721 | Pull-up resistor (R_PULLUP) | FIG. 29, FIG. 30 |
| 1722 | Capacitor (C0805C822KB RACTU) | FIG. 29 |
| 1723 | Capacitor for PPG sensor circuit | FIG. 30 |
| | (C_PPG_BULK2) | |
| 1724 | Pull-up resistor (R_PULLUP) | FIG. 30 |
| 1725 | Capacitor for PPG sensor circuit (C_PPG2) | FIG. 30 |
| 1726 | Capacitor (C0805C822KB RACTU) | FIG. 30 |

### DETAILED DESCRIPTION OF THE INVENTION

### I. Definitions

For the purposes of clarity and to ensure unambiguous claim construction, the following key terms are explicitly defined as they are used throughout this specification and the appended claims.
a. “Synergistic Fusion”: A computational process, typically executed by the processing unit (102), that combines data streams from disparate sensor classes (e.g., biomechanical, physiological, and biochemical) to generate a composite or diagnostic metric that provides a more holistic insight into a user's state than can be inferred from any single data stream alone.
b. “Alternating Actuation Mode”: A specific, power-efficient method of driving Electroactive Polymer (EAP) actuators (1402) wherein an initial high-voltage pulse (1602) of short duration is applied to initiate a physical deformation of the polymer, followed by a subsequent, lower-voltage or pulse-width modulated (1604) signal sufficient to sustain that deformation.
c. “High-Voltage Pulse” (1602): An electrical pulse of short duration and high voltage used to provide the initial energy to actuate and deform the EAP actuators (1402).
d. “PWM Signal” (1604): A relatively low-voltage, pulse-width modulated electrical signal used to provide the sustained energy required to hold the EAP actuators (1402) in a deformed state after the initial high-voltage pulse, significantly reducing overall power consumption.
e. “Sub-haptic”: A level of vibrotactile or mechanical stimulation, delivered by the active therapeutic system, that is below the threshold of the user's conscious perception but is sufficient to elicit a measurable physiological response in the autonomic nervous system.
f. “Biometric Signature”: A unique, multi-dimensional data template representing a physiological or behavioral characteristic of an individual. In a footwear embodiment, this may be a “Gait Signature”. In a smartwatch embodiment, this may be a “Cardiac Signature” derived from PPG waveform analysis.
g. “Trusted Execution Environment (TEE)”: A secure, hardware-isolated area on the main processor (SoC) where code and data are protected with respect to confidentiality and integrity, isolated from the rich operating system.

### II. The Symbiotic Wearable Platform: A Form-Factor-Agnostic Architecture

The present invention discloses a foundational technology platform that can be embodied in a plurality of wearable form factors. The core inventive concepts a self-sustaining, multi-source power system, a multi-modal sensor array for synergistic data fusion, and a hardware-secured processing architecture are not limited to a single application but constitute a versatile platform for advanced health monitoring.

### A. The Footwear Embodiment (Primary Embodiment)

In a first preferred embodiment, the symbiotic wearable platform is embodied in an article of footwear (100). This embodiment is particularly advantageous for applications that require detailed biomechanical data and active therapeutic intervention on the plantar surface of the foot.

### 1. Core Processing and Encapsulation

The platform employs a rigid-flex PCB architecture, comprising a main rigid PCB (202) connected to other flexible boards, such as a dorsal board (204) and a collar board (206). This rigid-flex architecture uses high-density FFC/FPC connectors that provide a reliable and flexible electrical interface between the main rigid PCB (202) and the other flexible boards (204, 206), which is crucial for the modular and ergonomic design of the platform. The physical connections between the rigid and flexible PCBs are robust and reliable to withstand the biomechanical stresses of walking and running. For example, the flexible PCBs are joined to the main rigid PCB (202) via high-density FFC/FPC connectors (306, 308) which are designed for high vibration resistance and durability. These connectors establish the Sensor Data Bus and System Control Busses that facilitate the seamless flow of data between the distributed units and the Main Hub.

Referring to FIGS. 1, 2, and 10, the smart footwear system (100) is architected around an advanced electronics core. The primary component is a processing unit (102), which in one embodiment is a System-on-Chip (SoC), such as the Nordic Semiconductor nRF5340. This SoC enables strategic “task partitioning” between a high-performance application core (102A) and a low-power network core (102B). This strategic task partitioning allows the high-performance core (102A) to handle computationally intensive tasks, such as running the deep learning fusion model, while the low-power network core (102B) can manage continuous, background tasks like sensor polling and wireless communication. This approach significantly optimizes power consumption, which is a critical technical enabler for the platform's ‘near-perpetual operation’ and distinguishes it from the prior art that relies on a single, less efficient processor core.

The core electronic components, including the processing unit (102) and the internal rechargeable battery (410), are housed within a hermetically sealed, water-resistant, and shock-resistant enclosure designed to meet or exceed the IP67 standard. The physical structure employs an innovative rigid-flex printed circuit board (PCB) design, comprising a main rigid PCB (202) housed in the arch of the shoe's midsole (210), electrically connected via flexible cables to a user-replaceable smart insole unit and other flexible PCBs, such as a dorsal board (204) and a collar board (206). Detailed layouts of the main rigid PCB, the flexible PCB for the instep and sides unit, and the flexible PCB for the rear collar unit are respectively shown in FIG. 28, FIG. 29, and FIG. 30.

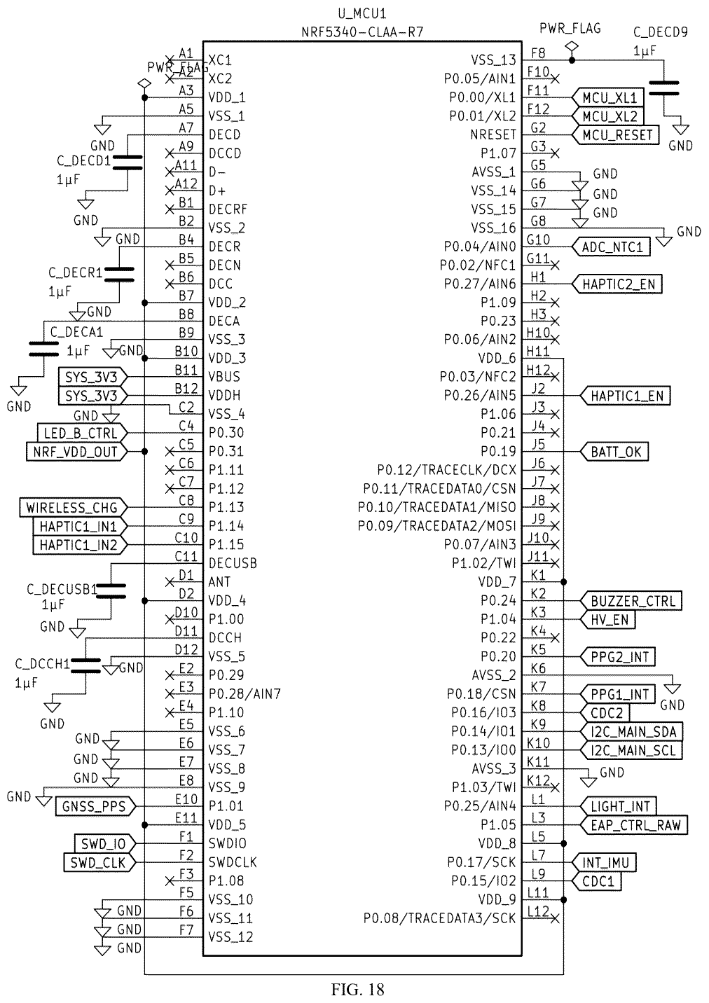

These figures provide a tangible representation of the physical implementation of the electronic architecture described herein. A detailed schematic of a preferred embodiment of the processing unit (U_MCU1) is shown in FIG. 18. Detailed layouts of the main rigid PCB, the flexible PCB for the instep and sides unit, and the flexible PCB for the rear collar unit are respectively shown in FIG. 28, FIG. 29, and FIG. 30. These figures provide a tangible representation of the physical implementation of the electronic architecture described herein.

### 2. Detailed Engineering of the Platform's Component Units

The footwear platform is comprised of several specialized electronic units that interconnect to form an integrated system.

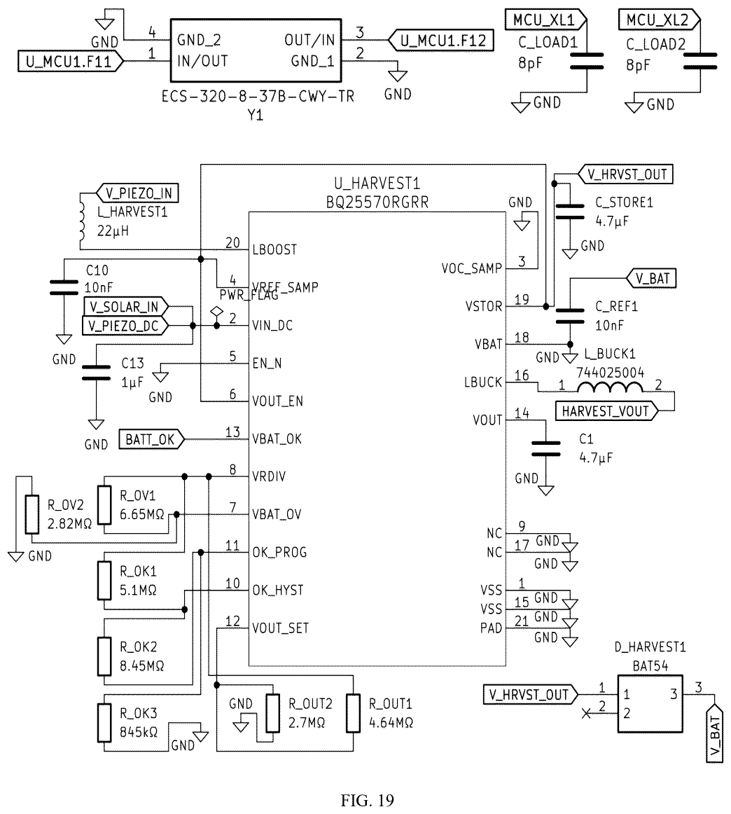

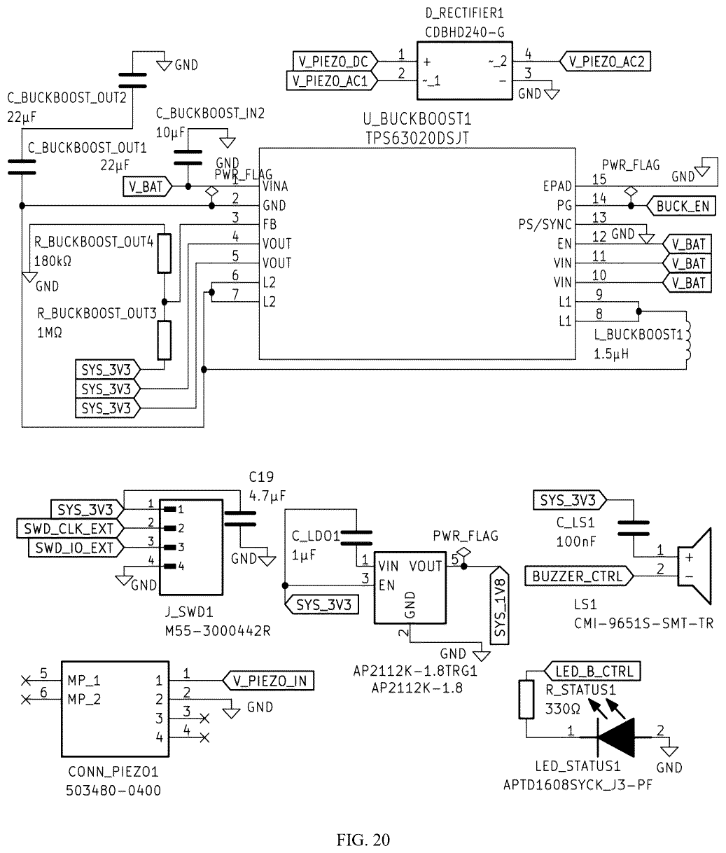

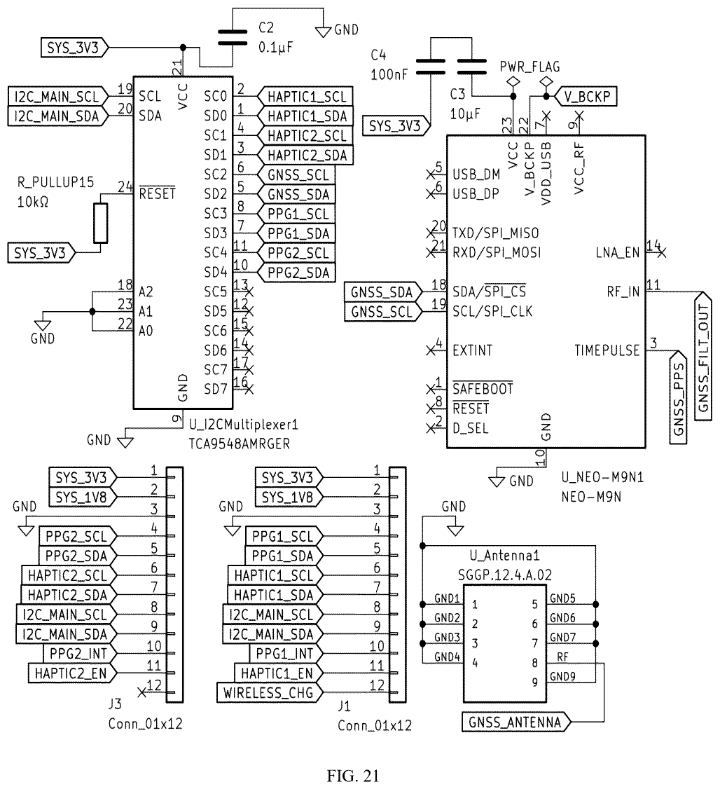

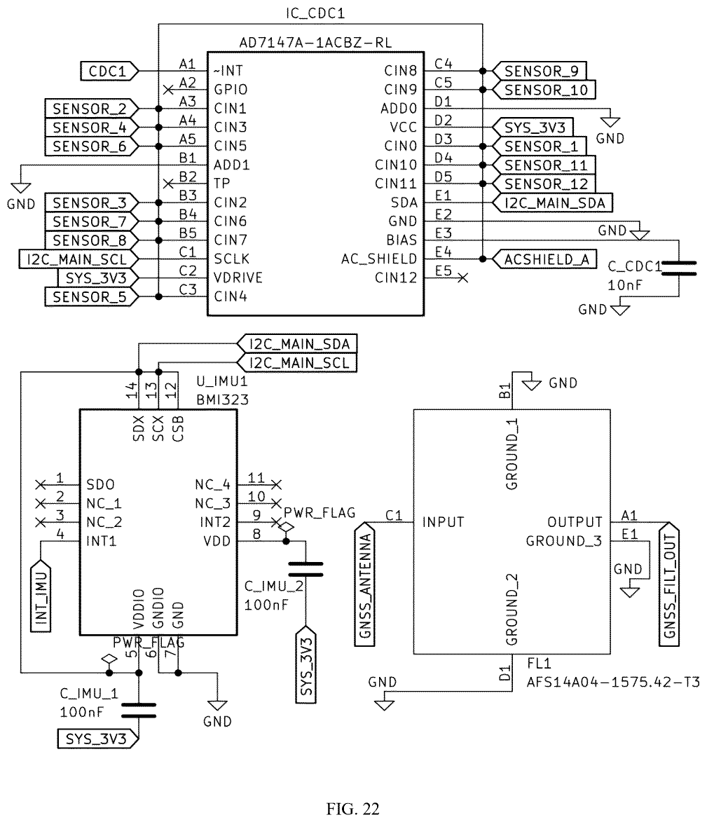

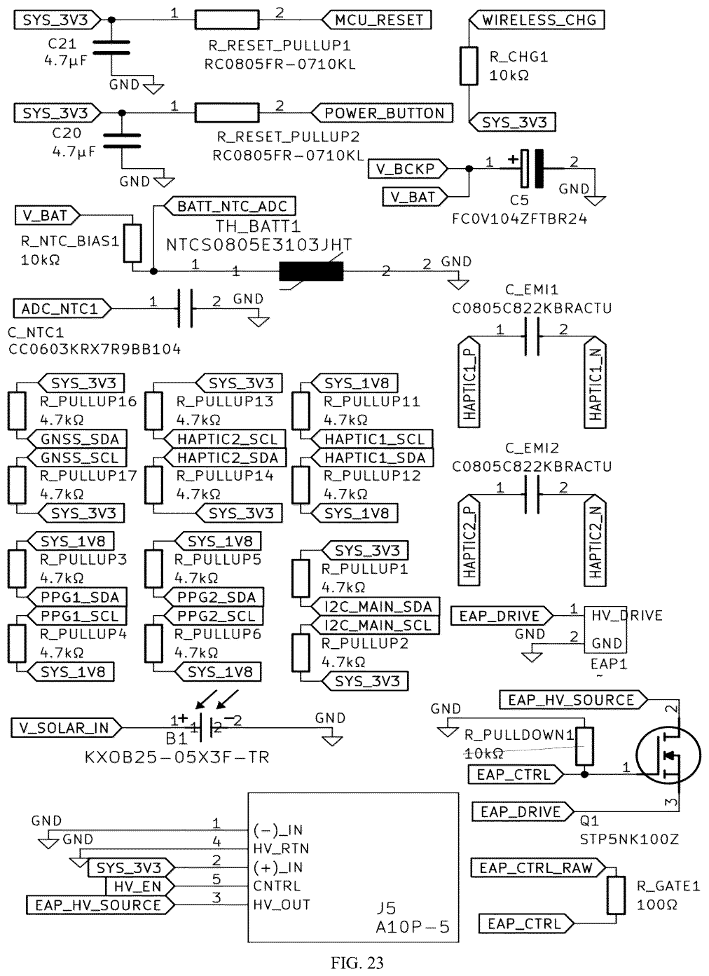

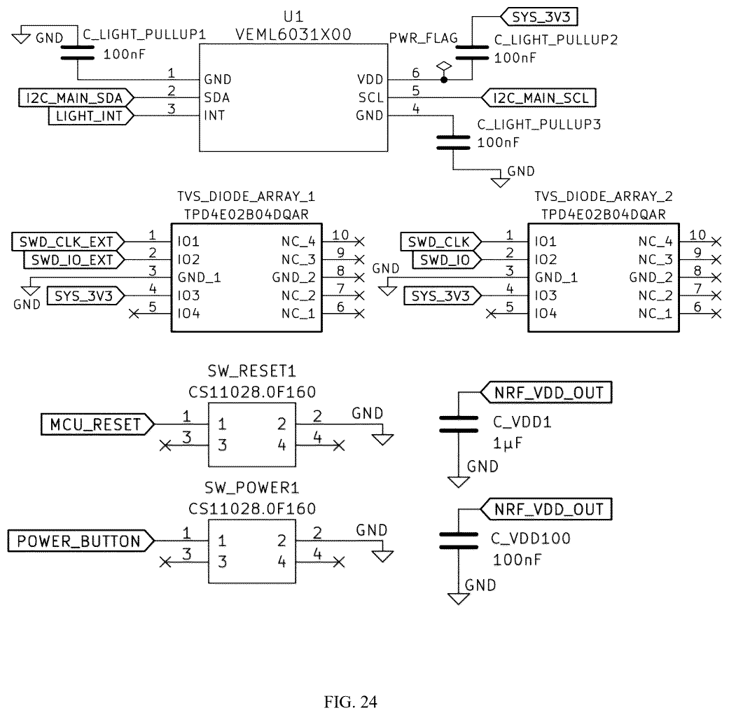

These units can be referenced in FIG. 2 and FIG. 15 by their numbers: The Main Hub (212), The Instep & Sides Unit (214), and The Rear Collar Unit (216).
a. The Main Hub (212): is designed with a plurality of physical interfaces, including a first high-density connector (302) that serves as the physical interface for the Sensor Data Bus, and a second high-density connector (304) for the System Control and Feedback Control Busses. This serves as the brain of the platform, containing the core processing, power management, and key sensor systems. As shown in the schematics, the Main Hub is composed of several detailed drawings. FIG. 18 shows the pinout and connections of the central processing unit (U_MCU1) of type Nordic Semiconductor nRF5340; FIG. 19 illustrates the synergistic, multi-source energy harvesting circuits; FIG. 20 details the power management, voltage regulation, and auxiliary power circuits; FIG. 21 illustrates the GNSS module (U_NEO-M9N1) and I2C multiplexer (U_I2CMultiplexer1); FIG. 22 shows the capacitive sensor (IC_CDC1) and the inertial measurement unit (U_IMU1); and FIG. 23 details the solar harvesting and user interface circuits.
The drawings further illustrate a variety of essential components. The schematic of the Tri-Hybrid Energy Harvesting system in FIG. 19 includes an inductor (L_HARVEST1) and a storage capacitor (C_STORE1) for the harvesting circuit. FIG. 20 shows an inductor (L_BUCKBOOST1) for the Buck-Boost converter and a piezoelectric sensor connector (CONN_PIEZO1).
The system also incorporates a debug and programming connector (J_SWD1) and a buzzer (LS1) for audible alerts, as shown in the same figure. FIG. 21 illustrates an I2C multiplexer (U_I2CMultiplexer1) to manage multiple sensor communication channels and a GNSS antenna filter (FL1).
FIG. 24 details an ambient light sensor (U1) and includes both power (SW_POWER1) and reset (SW_RESET1) push-button switches.
b. The Instep & Sides Unit (214): This distributed unit integrates key sensors and charging capabilities. The schematics show its components across two drawings.

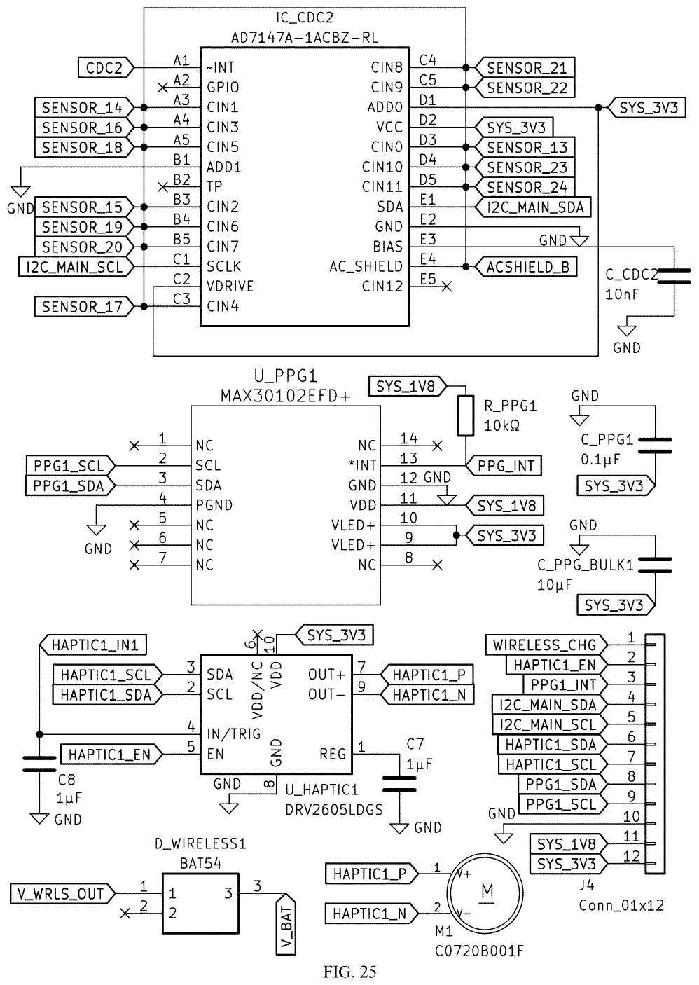

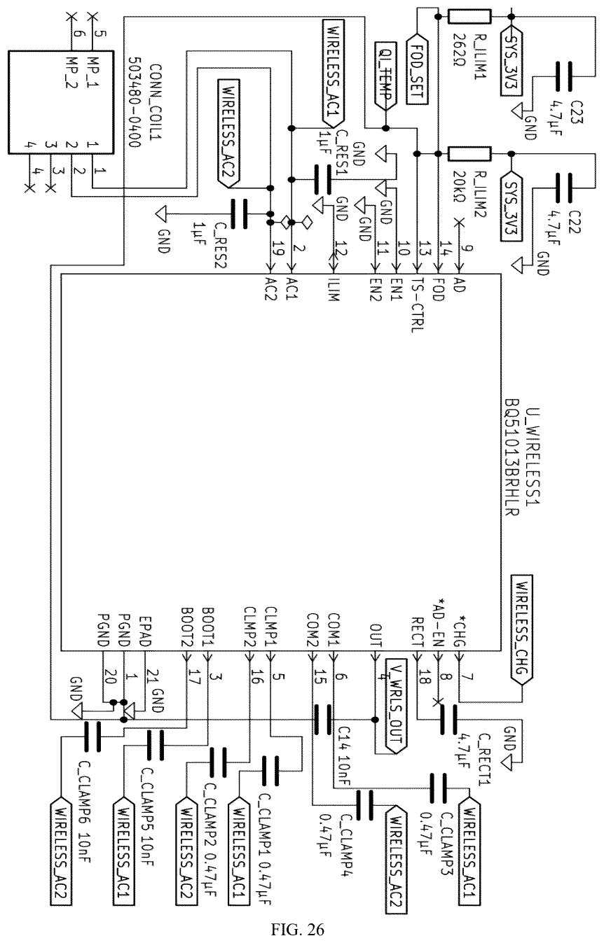

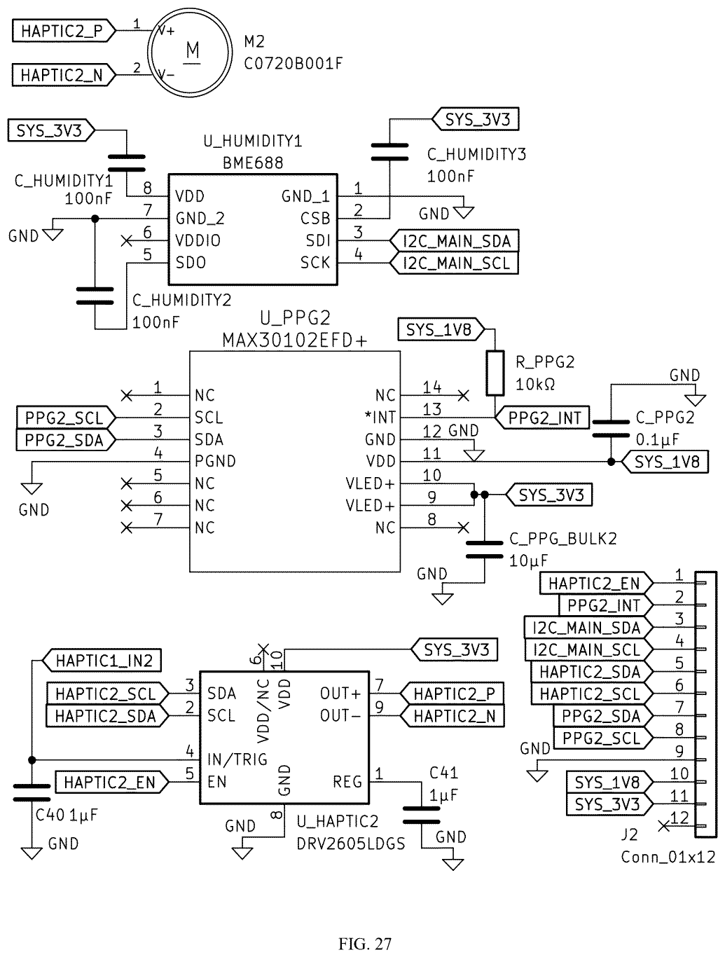

FIG. 24 and FIG. 26 illustrate the wireless power receiver (U_WIRELESS1) and its corresponding charging coil; and FIG. 25 illustrates the integration of a capacitive sensor (IC_CDC2), a photoplethysmography (PPG) sensor (U_PPG1), and a haptic driver (U_HAPTIC1).
c. The Rear Collar Unit (216): This distributed unit is represented by one detailed schematic, FIG. 27, which contains a haptic actuator (M2), a PPG sensor (U_PPG2), and an environmental sensor (U_HUMIDITY1) of type BME688.

TABLE 3
DETAILED COMPONENT LIST

| | | | | | |
|---|---|---|---|---|---|
| | | Value/ | | Manufacturer Part | Boards |
| Reference | Qty | Description | Manufacturer | Number (MPN) | Used In |

Integrated Circuits

| | | | | | |
|---|---|---|---|---|---|
| U_MCU1 | 1 | nRF5340 SoC, | Nordic | NRF5340-CLAA-R7 | Main |
| | | Dual-Core, BLE | Semiconductor | | Hub |
| U_HARVEST1 | 1 | Piezoelectric | Texas | BQ25570RGRR | Main |
| | | Energy | Instruments | | Hub |
| | | Harvester | | | |
| U_WIRELESS1 | 1 | Qi Wireless | Texas | BQ51013BRHLR | Instep |
| | | Power Receiver | Instruments | | &Sides |
| | | | | | Unit |
| U_BUCKBOOST1 | 1 | Buck-Boost | Texas | TPS63020DSJT | Main |
| | | Converter, 4A | Instruments | | Hub |
| U_LDO1 | 1 | LDO Regulator, | Diodes Inc. | AP2112K-1.8TRG1 | Main |
| | | 1.8 V | | | Hub |
| U_I2CMUX1 | 1 | −8Channel I2C | Texas | TCA9548AMRGER | Main |
| | | Multiplexer | Instruments | | Hub |
| U_GNSS1 | 1 | GNSS Module | u-blox | NEO-M9N-00B | Main |
| | | | | | Hub |
| U_IMU1 | 1 | −6Axis IMU | Bosch | BMI323 | Main |
| | | | Sensortec | | Hub |
| U_PPG1 | 1 | PPG Sensor | Analog | MAX30102EFD++ | Instep |
| | | (Instep) | Devices | | &Sides |
| | | | | | Unit |
| U_PPG2 | 1 | PPG Sensor | Analog | MAX30102EFD+ | Rear |
| | | (Collar) | Devices | | Collar |
| | | | | | Unit |
| U_ENV1 | 1 | Environmental | Bosch | BME688 | Rear |
| | | | | | Collar |
| | | Sensor | Sensortec | | Unit |
| IC_CDC1 | 1 | Capacitive | Analog | AD7147A-1ACBZ- | Main |
| | | Sensor Ctrl | Devices | RL | Hub |
| | | (Insole) | | | |
| IC_CDC2 | 1 | Capacitive | Analog | AD7147A-1ACBZ- | Instep |
| | | Sensor Ctrl | Devices | RL | &Sides |
| | | (Instep) | | | Unit |
| U_ALS1 | 1 | Ambient Light | Vishay | VEML6031X00 | Main |
| | | Sensor | | | Hub |
| U_HAPTIC1 | 1 | Haptic Driver | Texas | DRV2605LDGS | Instep |
| | | (Instep) | Instruments | | &Sides |
| | | | | | Unit |
| U_HAPTIC2 | 1 | Haptic Driver | Texas | DRV2605LDGS | Rear |
| | | (Collar) | Instruments | | Collar |
| | | | | | Unit |

Connectors

| | | | | | |
|---|---|---|---|---|---|
| CONN_COIL1 | 1 | −4Pin FFC/FPC | Molex | 0400-503480 | Instep |
| | | Connector | | | &Sides |
| | | | | | Unit |
| CONN_PIEZO1 | 1 | −4Pin FFC/FPC | Molex | 0400-503480 | Main |
| | | Connector | | | Hub |
| J_HUB_INSTEP | 1 | −12Pin FFC/FPC | Hirose | FH12-12S-0.5SH(55) | Main |
| | | Connector | | | Hub |
| J_HUB_COLLAR | 1 | −12Pin FFC/FPC | Hirose | FH12-12S-0.5SH(55) | Main |
| | | Connector | | | Hub |
| J_INSTEP_HUB | 1 | −12Pin FFC/FPC | Hirose | FH12-12S-0.5SH(55) | Instep |
| | | Connector | | | &Sides |
| | | | | | Unit |
| J_COLLAR_HUB | 1 | −12Pin FFC/FPC | Hirose | FH12-12S-0.5SH(55) | Rear |
| | | Connector | | | Collar |
| | | | | | Unit |
| J_SWD1 | 1 | SWD | Harwin | M55-3000442R | Main |
| | | Programming | | | Hub |
| | | Connector | | | |

Other Components

| | | | | | |
|---|---|---|---|---|---|
| FL_GNSS1 | 1 | SAW Filter | Abracon | AFS14A04-1575.42- | Main |
| | | | | T3 | Hub |
| LED_STATUS1 | 1 | Yellow-Green | Kingbright | APTD1608SYCK/J3- | Main |
| | | SMD LED | | PF | Hub |
| LS1 | 1 | Magnetic Buzzer | CUI Devices | CMI-9651S-SMT-TR | Main |
| | | | | | Hub |
| M1 | 1 | Vibration Motor | Jinlong | C0720B001F | Instep |
| | | (Instep) | Machinery | | &Sides |
| | | | | | Unit |
| M2 | 1 | Vibration Motor | Jinlong | C0720B001F | Rear |
| | | (Collar) | Machinery | | Collar |
| | | | | | Unit |
| SW_POWER1 | 1 | Push Button | C&K | KSC201J LFS | Main |
| | | Switch | | | Hub |
| SW_RESET1 | 1 | Push Button | C&K | KSC201J LFS | Main |
| | | Switch | | | Hub |
| Y1 | 1 | 32.768 kHz | ECS Inc. | ECS-320-8-37B- | Main |
| | | Crystal | | CWY-TR | Hub |
| U_Antenna1 | 1 | GNSS Patch | Taoglas | SGGP.12.4.A.02 | Main |
| | | Antenna | Limited | | Hub |

TABLE 4
MAPPING INVENTIVE SOLUTIONS TO TECHNICAL COMPONENTS

| Key Components | Inventive Technical | |
|---|---|---|
| (Reference Numbers) | Solution | Problem Addressed |
| 406, 404, 402, 400 | Synergistic Tri-Hybrid Power | Power Dependency & Data |
| | System | Gaps |
| 508, 506, 504, 502, 500 | Multi-Modal Sensor Array & | Limited Sensory Scope |
| | Synergistic Fusion | |
| 1408, 1406, 1402, 650 | Active Therapeutic System | Passive Monitoring Paradigm |
| | (Closed-Loop) | |
| 102, 102A, 1322 | Hardware-Secured Biometric | Software-Level Security |
| | Authentication | Risks |
| 1502, 508, 100 | Modular Architecture & | Short Lifecycles & E-Waste |
| | Material Degradation Sensing | |

### 3. The Synergistic Tri-Hybrid Power System (400)

A core pillar of the invention is the synergistic tri-hybrid power system (400). This system provides a direct solution to the power dependency problem by managing three integrated energy-harvesting sources: a solar/photovoltaic harvesting circuit (402) for direct sunlight and ambient light, a kinetic/piezoelectric harvesting circuit (404), and a wireless/inductive charging circuit (406). The system's power management subsystem is designed to intelligently prioritize power drawing from the renewable harvesting sources (solar and kinetic) before utilizing the internal rechargeable battery (410). This approach not only ensures continuous data collection but also extends the lifespan of the battery by reducing the frequency of deep charge and discharge cycles. The selection of specific components, such as the TI BQ25570RGRR (408) with its ultra-low quiescent current and cold-start capability, is a critical technical enabler for the “near-perpetual operation” claim. This particular selection is not merely a component choice but a fundamental technical enabler.

The TI BQ25570RGRR (408), with its ultra-low quiescent current of less than 500 nA, is the enabling factor that permits continuous, background operation of power-intensive sensor modalities, such as the biochemical gas sensor (508). This synergistic pairing creates a novel method for material degradation sensing that was not feasible in prior art devices that only focused on extending battery life. This direct causal relationship between the energy-saving solution and the energy-consuming solution demonstrates that the invention is not a simple aggregation of known parts, but a unique, non-obvious combination that solves a foundational technical problem.

TABLE 5
TECHNICAL SPECIFICATIONS OF THE TRI-HYBRID POWER SYSTEM

| Component | Key Datasheet Features | Role in System Sustainability |
|---|---|---|
| TI BQ25570RGRR | Cold-Start Voltage: ≥600 | Enables continuous energy harvesting |
| (408) | mV | from low-power, intermittent sources, |
| | Quiescent Current: 488 nA | ensuring the device remains |
| | (typical) | operational even in non-ideal |
| | Efficiency: up to 93% (boost) | conditions, such as dim ambient light |
| | | or with minimal motion. |
| TI BQ51013BRHLR | Peak AC-DC Efficiency: | Provides a highly efficient and |
| (406) | 93% | reliable wireless charging option as a |
| | WPC v1.0 Compliant | robust backup to the harvesting |
| | Communication | sources. |
| TI TPS63020DSJT | Input voltage: 1.8 V to 5.5 V | A high-efficiency buck-boost |
| | Output: up to 2 A | converter that ensures stable power |
| | | delivery to all subsystems regardless |
| | | of the input source or battery state. |

### 4. The Distributed Multi-Modal Sensor Array and Synergistic Fusion (500)

The platform directly addresses the problem of a constrained sensory scope by holistically and synergistically integrating diverse sensor modalities that the prior art has failed to combine effectively due to power limitations. For instance, the algorithm can generate an accurate, cuffless blood pressure estimate by analyzing the morphology of the PPG waveform, achieving a standard deviation of error (SDE) of 4.8 mmHg for Systolic Blood Pressure (SBP).

This exemplifies an “unexpected result” that strengthens the non-obviousness argument. The true innovation lies in the “data-level fusion” approach executed by the processing unit (102), which goes beyond merely displaying multiple data points. The innovation is that the deep learning model, specifically a Long Short-Term Memory (LSTM) network, does not merely process each data stream independently, but rather integrates them synergistically.

The LSTM network is specifically trained on time-series data from the different sensors, with each time step's input vector containing a concatenation of the synchronized data from the IMU (502), PPG (506), and biochemical sensor (508). The network is configured to learn complex, non-linear dependencies between these inputs over time, allowing it to output a composite health metric or prediction, such as a cuffless blood pressure estimate, which is a result of the synergistic fusion of the data streams.

The network leverages its superior ability to analyze complex temporal patterns to identify non-linear correlations between biomechanical data (such as motion and gait signature data from the IMU) and physiological data (such as the PPG waveform morphology). This intricate fusion enables the system to generate accurate, composite health metrics, such as a cuffless blood pressure estimate, a result that could not have been achieved by analyzing any of the data streams individually. This ability to deduce a new physiological indicator by fusing disparate data sources constitutes a unique technical solution that clearly distinguishes the invention from the prior art.

The platform integrates a comprehensive sensor array (500) designed not just to collect data, but to fuse it in a synergistic manner for a richer understanding of the user's state.

The array includes Biomechanical Sensors, such as a high-precision Inertial Measurement Unit (IMU) (502), Physiological Sensors, such as at least one Photoplethysmography (PPG) sensor (506). and a Biochemical Sensor, such as an environmental gas sensor (508) configured to detect volatile organic compounds (VOCs).

The synergistic relationship extends beyond mere power management and data aggregation to a direct causal link between components. The power management IC (408), with its ultra-low quiescent current of less than 500 nA, serves as a fundamental enabler for advanced sensor modalities that were previously unfeasible due to power limitations. For example, this energy efficiency permits the continuous, background operation of a biochemical gas sensor (508) that is configured to detect a volatile organic compound signature. This combination is not obvious, as the power management IC's unique characteristics enable the biochemical sensor to be a constant, active part of the system, thereby creating the novel method for material degradation sensing. This synergistic pairing directly solves a long-felt, unsolved technical problem by providing a continuous data stream from a power-intensive sensor, which was not achievable in the prior art that only focused on extending battery life.

TABLE 6
TECHNICAL SPECIFICATIONS AND SYNERGISTIC
VALUE OF KEY SENSORS

| | Specific | Key Technical | Contribution to |
|---|---|---|---|
| Sensor Type | Component | Features | Synergistic Fusion |
| Biomechanical | Bosch BMI323 | 16-bit resolution, 790 | Provides critical contextual |
| | (IMU) (502) | μA current | data on user activity and |
| | | consumption, | motion, enabling the |
| | | intelligent motion- | system to accurately |
| | | triggered interrupts | interpret physiological and |
| | | | biochemical readings. |
| Physiological | Maxim MAX30102 | High SNR, robust | Gathers accurate and |
| | (PPG) (506) | motion artifact | reliable vital signs data |
| | | resilience, ambient | even during movement, |
| | | light rejection | which is then fused with |
| | | | biomechanical data for a |
| | | | contextualized health |
| | | | assessment. |

### B. The Smartwatch Embodiment (Alternative Embodiment)

In a second preferred embodiment, the symbiotic wearable platform is embodied in a smartwatch. This embodiment leverages the same core principles but adapts them to a different form factor. The synergistic, multi-source power system (400) is adapted for this form factor, wherein the solar energy harvesting circuit (402) may be integrated into the watch face or band, and the kinetic energy harvesting circuit (404) is configured to generate power from arm motion. The multi-modal sensor array (500) is also adapted for wrist-based sensing, including an IMU (502), a multi-wavelength PPG sensor (506), and a biochemical sensor (508) configured to detect biomarkers in sweat.

### III. Hardware-Fortified Biometric Security Architecture

A cornerstone of the platform, regardless of its embodiment, is its novel approach to biometric security. The application core (102A) of the processing unit (102) features a hardware-isolated Trusted Execution Environment (TEE). This enables a highly secure method for biometric authentication, referred to as the “Biometric Vault,” which protects sensitive user data at the hardware level, a significant improvement over software-level security.

The central innovation lies in the fact that the entire authentication process occurs entirely within the secure, hardware-enforced confines of the TEE, which prevents exposure to software-based attacks. A user's unique “Biometric Signature” (e.g., a gait pattern from the biomechanical sensors) is processed, and a secure template is generated and stored within the TEE.

When an authentication request is received, real-time biometric data is fed to a specialized machine learning model, such as a Siamese Neural Network, operating within the TEE. The Siamese Neural Network architecture is uniquely suited to this task, as it is configured to compare two inputs, in this case the live biometric data and the stored template, and determine their degree of similarity or “match score” entirely within the secure environment.

This specific combination of a Siamese Neural Network operating entirely within a TEE is a key inventive step that distinguishes the invention from the prior art, which either lacks hardware isolation for biometric processing or does not use this specific deep learning model for gait verification. This ensures that no sensitive biometric data is ever exposed to the non-secure operating system, which is where most software-based attacks occur.

The use of a Siamese Neural Network is a key inventive step, as its architecture is uniquely suited to verify the subtle and dynamic patterns of a user's gait, providing a robust and fault-tolerant authentication that is highly resistant to spoofing or impersonation attacks.

The comparison between the real-time data and the stored template is performed entirely within the TEE's isolated environment. This ensures that no sensitive biometric data is ever exposed to the non-secure operating system, which is where most software-based attacks occur.

As shown in FIG. 13, the biometric authentication process begins with the collection of real-time gait data (1302) in the normal world (1320). Upon receiving an authentication request (1304), the system transitions to the secure world (1322) within the TEE. A secure template is loaded from memory (1306), and a comparison is performed entirely within this isolated environment (1308) to generate an authentication result (1312), which is then returned to the normal world (1320).

TABLE 7
BIOMETRIC VAULT WORKFLOW & HARDWARE-
SOFTWARE INTERACTION

| Step in the “Biometric | Physical Components | |
|---|---|---|
| Vault” Process | Involved | Specific Technical Benefit |
| 1. Template Generation | System-on-a-Chip (SoC) with | The biometric template is |
| Storage | TEE, Secure Memory | generated and stored in a |
| | | hardware-isolated |
| | | environment, preventing |
| | | exposure to malware. |
| 2. Secure Data Ingestion | Biometric Sensor Array, | Sensitive data is transferred |
| | Secure Bus Interface, TEE | directly to the secure |
| | | environment, bypassing the |
| | | untrusted rich operating |
| | | system. |
| 3. Real-Time Verification | Specialized Machine | The comparison between live |
| | Learning Model (e.g., | data and the stored template |
| | Siamese Neural Network) | is performed in a partitioned |
| | operating within the TEE | environment, ensuring |
| | | sensitive data is never |
| | | exposed to the non-secure |
| | | operating system. |
| 4. Authentication Output | TEE, Cryptographic Unit | The authentication result is |
| | | provided as an encrypted |
| | | signal, offering a high level |
| | | of security against software- |
| | | based attacks. |

### IV. The Active Therapeutic and Closed-Loop Control System

In certain embodiments, the platform includes an active therapeutic system with an array of Electroactive Polymer (EAP) actuators (1402). The EAP actuators (1402) are integrated into a layered insole structure, positioned beneath a top fabric layer (1404) and above a flexible substrate (806). They are arranged in a specific array to target key reflexology points or to provide gait correction across the plantar surface of the foot. These actuators are electrically connected to the High-Voltage Driver Circuit (1406) via durable and flexible embedded traces within the flexible PCB, ensuring that the therapeutic intervention can be delivered precisely and reliably.

This inventive method exploits a key property of dielectric EAPs: their ability to maintain a physical deformation under a DC voltage with minimal power consumption. Instead of constantly applying a high voltage, the system sends a short, high-voltage pulse (1602) to initiate the desired deformation, then switches to a subsequent, lower-voltage or pulse-width-modulated (1604) signal sufficient to sustain the deformation. This significantly reduces energy consumption, making continuous therapeutic intervention in a wearable device practical and effective.

The active therapeutic system utilizes an array of Electroactive Polymer (EAP) actuators (1402) which are driven by a specialized High-Voltage Driver Circuit (1406) that includes a High-Voltage Boost Converter (1408).

It is important to note that while the “Alternating Actuation Mode” effectively manages power consumption, dielectric elastomer actuators are known to exhibit “poor lifetime characteristics” due to a phenomenon known as “dielectric breakdown” when subjected to a high voltage over time. The present invention's use of a short, high-voltage pulse followed by a low-voltage sustaining signal mitigates this inherent risk by minimizing the duration of high-stress electrical fields across the polymer. This approach directly addresses and provides a technical solution to the long-term reliability problem associated with this class of actuators.

It must be emphasized that the synergistic tri-hybrid power system is the fundamental technical enabler that makes the active therapeutic system practically viable in a wearable device. While EAP actuators require short, high-voltage pulses, they also need sustained power to maintain their active state. Due to its ultra-efficient design with a low quiescent current (488 nA), the power system ensures that a sufficient energy budget is always available to operate the actuators without compromising the comprehensive sensing capabilities. This direct causal relationship between the energy-saving solution (the power system) and the energy-consuming solution (the actuators) highlights the synergistic nature of the invention and demonstrates that it solves a foundational technical problem that was previously unsolved in prior art devices.

This system operates in a closed loop, where data from a plantar pressure sensing array (504) is continuously analyzed by the processing unit (102). If the system detects a gait anomaly, it can immediately activate the EAP actuators to provide a corrective force on the plantar surface of the foot, adjusting the user's gait in real-time. The physical actuation of the EAP actuators requires a specialized High-Voltage Driver Circuit (1406), which includes a High-Voltage Boost Converter (1408) and a High-Voltage MOSFET (Q1).

The closed-loop control is further enhanced by a Capacitance Measurement Circuit (1410) that is configured to measure the change in capacitance of the EAP actuators (1402) as they deform. This direct feedback mechanism is crucial for the platform's ability to compensate for the inherent non-linear properties and material drift of the EAP actuators, thereby enabling the precise and repeatable therapeutic intervention required for clinical applications. This capacitance measurement provides a direct feedback signal to the processing unit (102), enabling it to dynamically adjust the applied voltage to achieve a precise target force or displacement, thereby compensating for the material's inherent non-linear properties.

### V. Inventive Methods of Operation and Advanced Applications

The synergistic fusion algorithm, illustrated in the flowchart of FIG. 12, begins with the acquisition of data from biomechanical sensors (1202) and synchronized streams from biochemical sensors (1204). The processing unit then calculates a composite risk score (1206) based on these fused data streams. If the risk score exceeds a predefined threshold (1208), the system triggers a high-priority alert to the user and clinician (1210).

This network represents the highest expression of the symbiotic concept, extending from the interdependence of internal components to the synergy of the community, creating a data ecosystem of mutual benefit for all users.

The unique synergistic combination of hardware systems, secure architecture, and AI models enables several novel methods of operation that provide a significant technical improvement over the prior art.
a. Decentralized Health Mesh Network: A future embodiment of the device could function as a node in a secure, decentralized mesh network. This would allow a group of devices to share anonymized health data directly with each other, creating a real-time collective monitoring system without reliance on a centralized server. This decentralized approach enhances user privacy and provides a robust, fault-tolerant system for community health monitoring.
b. Enhancing Product Lifecycle Sustainability: The platform directly addresses the e-waste crisis by incorporating a method for monitoring material degradation. The biochemical gas sensor (508) is uniquely configured to detect specific “chemical fingerprints” released by materials during degradation. By correlating this data with performance metrics from the biomechanical sensors, the system can predict component failure and alert the user to replace a specific part rather than the entire device, which significantly extends the product's lifespan and reduces its environmental impact. This approach is supported by research showing that self-healing polymers, which could be integrated into the device, can recover up to 90% of their original strength and lead to a 30-50% reduction in carbon emissions over the product's lifecycle. This method not only reduces e-waste, but it empowers the user by giving them the tools and information necessary to be an active participant in their product's longevity, granting them personal agency against the planned obsolescence cycles that characterize the industry.
c. Expanded Scope of Clinical and Lifestyle Applications: The platform is configured to monitor, analyze, and provide interventions for a plurality of conditions and applications, including but not limited to: Clinical and Pathological Monitoring (e.g., diabetic foot ulcers, peripheral neuropathy, Parkinson's disease), Rehabilitation and Performance, Safety and Lifestyle (e.g., fall detection, geo-fencing), and Women's Health (e.g., menstrual cycle tracking, preeclampsia monitoring).

### VI. Exemplary Embodiments and Non-Limiting Language

It is to be understood that while specific, commercially available components (e.g., integrated circuits from Nordic Semiconductor, Texas Instruments, or Bosch) have been identified in this specification for the purpose of providing a clear and enabling disclosure, the invention is not limited to these specific components. Any other components, now known or later developed, that perform an equivalent function are considered to be within the scope of this invention. The detailed descriptions of specific embodiments, such as footwear and smartwatch, are provided as examples and are not intended to be limiting. This critical defensive measure ensures that the patent protects the underlying inventive concepts, regardless of the specific components used in a final product, thereby preventing competitors from simply swapping out a component to circumvent the patent.

### VII. Method for Mental State Modulation

This method provides a technical solution for stress modulation. The system is configured to detect a physiological state indicative of stress by monitoring biomarkers such as a sustained decrease in heart rate variability (HRV) via the PPG sensor (506). In response, the processing unit (102) controls the active therapeutic system to deliver rhythmic, sub-sensory haptic signals through the EAP actuators (1402) to the user's foot, effectively promoting a state of calmness.

### VIII. Method for Non-invasive Biochemical Infection Detection

This method leverages the biochemical gas sensor (508) and is depicted in FIG. 12. The method comprises creating a user-specific baseline profile for Volatile Sulfur Compounds (VSCs), which are biomarkers of infection. An alert is generated if a subsequently measured VSC concentration exceeds the user's pre-established baseline by a predetermined threshold.

### IX. Expanded Scope of Inventive Applications

a. Expanded Safety and Lifestyle Features: The platform's capabilities are extended to include additional safety and lifestyle applications. The processing unit (102) is configured to provide haptic navigational guidance, wherein directional cues are provided via patterned vibrations through the haptic actuators (602, 612).
b. Future Embodiments and Wellness Applications: The disclosed architecture is configured to support future technological integrations and applications. The invention provides a method for automated reflexology. The processing unit (102) is configured to store a digital map of reflexology points and control the active therapeutic system (1402) to apply targeted, patterned, and timed pressure to these points, providing an automated wellness session in response to a user command or a detected physiological state such as stress.

### X. A User Empowerment and Personal Agency

Unlike the prior art's passive monitoring paradigm, this invention represents a paradigm shift that places the user in a position of power. Instead of being a mere source of data analyzed remotely, the user becomes an active and empowered partner in their own health management. The closed-loop control system, which uses EAP actuators (1402) to provide real-time gait correction based on data from the plantar pressure sensing array (504), is a direct embodiment of this empowerment. It does not just collect data, but uses it to effect an immediate and tangible change in the user's function, giving them a sense of control over their health and well-being.

### XI. Method for Environmental and Interactive Connectivity

The platform is further configured to interact with its environment, transforming it into an active node in an interconnected ecosystem. The processing unit (102), utilizing its network core (102B) and alternative communication protocols such as Wi-Fi, is configured to establish communication with external systems, beacons, and displays.

This enables a plurality of interactive applications, including, but not limited to: in a marathon or sporting event, the system can transmit real-time performance data to a nearby display as the user passes a checkpoint; (2) in an interactive public space or entertainment venue, the system can communicate with environmental controls to influence ambient lighting or music based on the user's biometric data; (3) in a transit system, the system can perform automated and secure electronic ticketing by communicating with station gates, with the transaction secured by the Trusted Execution Environment (TEE); and (4) in professional sports, the system's unique identifier can be used by automated camera systems for real-time player tracking.

What is claimed is:

1. A wearable electronic device, comprising:
a. a processing unit (102);
b. an internal rechargeable battery (410);
c. a multi-source power system (400) operatively connected to the processing unit (102) and the internal battery (410), said power system (400) comprising at least two distinct energy harvesting modalities selected from the group consisting of a solar circuit (402), a kinetic circuit (404), and a wireless charging circuit (406);
d. wherein said power system (400) includes a power management IC (408) having a quiescent current of less than 500 nA, said IC specifically configured to enable the continuous, background operation of the at least one biochemical sensor (508) within the multi-modal sensor array;
e. and the multi-modal sensor array (500) operatively connected to the processing unit (102), said sensor array (500) comprising at least one biomechanical sensor (502), at least one physiological sensor (506), and at least one biochemical sensor (508);
f. wherein the processing unit (102) is configured to synergistically fuse data from said multi-modal sensor array (500) to generate a composite health metric.

2. A wearable electronic device, comprising:
a. a processing unit (102) having a hardware-isolated trusted execution environment (TEE);
b. wherein the processing unit (102) is configured to execute a biometric authentication method using a Siamese Neural Network to compare a biometric signature template to real-time sensor data, wherein the entire process is performed within the trusted execution environment (TEE).

3. A wearable electronic device, comprising:
a. a processing unit (102);
b. an array of Electroactive Polymer (EAP) actuators (1402) operatively connected to the processing unit (102);
c. a high-voltage driver circuit (1406) configured to actuate the EAP actuators (1402);
d. and a multi-source power system (400) operatively connected to the driver circuit (1406);
e. wherein the processing unit (102) is configured to provide a closed-loop therapeutic intervention by activating the EAP actuators (1402) using an alternating actuation mode, said mode comprising applying a high-voltage pulse (1602) to initiate deformation followed by a lower-voltage signal (1604) to sustain deformation.

4. A method for providing a composite health assessment via a wearable electronic device, the method comprising the steps of:
a. collecting, via a multi-modal sensor array (500) of the wearable electronic device, a plurality of data streams including a first data stream from at least one biomechanical sensor (502), a second data stream from at least one physiological sensor (506), and a third data stream from at least one biochemical sensor (508);
b. processing, by a processing unit (102) of the wearable electronic device, the data streams using a synergistic fusion algorithm that employs a deep learning model;
c. and generating, by the processing unit (102), a composite health metric based on the identified cross-modal patterns.

5. A method for providing power-efficient actuation in a wearable electronic device, the method comprising the steps of:
a. applying, by a driver circuit (1406), a high-voltage pulse (1602) of a short duration to an Electroactive Polymer (EAP) actuator (1402) to initiate a physical deformation of the actuator;
b. and applying, by the driver circuit (1406), a lower-voltage signal (1604) to the EAP actuator (1402) to sustain the physical deformation with reduced power consumption.

6. A method for enhancing product lifecycle sustainability of a wearable electronic device, the method comprising the steps of:
a. detecting, via a biochemical gas sensor (508), a volatile organic compound signature indicative of a material degradation of a component of the wearable device;
b. collecting, via at least one biomechanical sensor (502), biomechanical data indicative of a functional performance of the component;
c. correlating, by a processing unit (102), the detected volatile organic compound signature with the collected biomechanical data;
d. and generating an alert indicative of a need for component replacement based on said correlation.

7. A method for providing hardware-secured biometric authentication in a wearable device, the method comprising the steps of:
a. collecting, via one or more sensors of the wearable device, real-time biometric data of a user;
b. in response to an authentication request received in a normal processing environment of a processing unit (102), transitioning to a secure processing environment of the processing unit (102), wherein the secure processing environment is a hardware-isolated trusted execution environment (TEE);
c. within the secure processing environment: loading a stored biometric signature template from a secure memory;
d. comparing the real-time biometric data to the stored biometric signature template to generate a match score;
e. and generating an authentication result based on the match score;
f. and returning the authentication result from the secure processing environment to the normal processing environment.

8. The wearable electronic device of claim 1, wherein the at least one biochemical sensor (508) is a gas sensor configured to detect a volatile organic compound signature indicative of material degradation of a component of the wearable device.

9. The wearable electronic device of claim 1, wherein the multi-source power system (400) further comprises a wireless charging circuit (406).

10. The wearable electronic device of claim 1, wherein the device is embodied in an article of footwear (100) and wherein the at least one biomechanical sensor comprises a plantar pressure sensing array (504).

11. The wearable electronic device of claim 2, wherein the biometric authentication method uses a Siamese Neural Network operating within the TEE to compare the biometric signature template to the real-time sensor data.

12. The wearable electronic device of claim 2, wherein the device is an article of footwear (100) and the real-time biometric data comprises gait data collected from at least one biomechanical sensor (502, 504).

13. The wearable electronic device of claim 3, wherein the closed-loop therapeutic intervention further comprises:
a. a capacitance measurement circuit (1410) configured to measure a change in capacitance of the EAP actuators (1402) as the actuators deform;
b. and the processing unit (102) configured to dynamically adjust an applied voltage to the EAP actuators (1402) based on said change in capacitance to achieve a targeted force or displacement profile.

14. The wearable electronic device of claim 3, wherein the device is an article of footwear (100) and the at least one sensor is a plantar pressure sensing array (504), and wherein the therapeutic intervention is a real-time gait correction.

15. The method of claim 4, wherein the deep learning model is a Long Short-Term Memory (LSTM) network configured to process time-series data from the multi-modal sensor array.

16. The method of claim 4, wherein the composite health metric is a cuffless blood pressure estimate derived from an analysis of a photoplethysmography (PPG) waveform morphology fused with biomechanical contextual data.

17. The method of claim 5, further comprising:
a. measuring a change in capacitance of the EAP actuator (1402) as it deforms;
b. and dynamically adjusting the applied lower-voltage signal (1604) based on said measured change in capacitance to achieve a targeted force or displacement.

18. The method of claim 6, wherein said volatile organic compound signature is detected by a gas sensor with a low-power sensing capability enabled by a multi-source power system (400) having a power management IC with a quiescent current of less than 500 nA.

19. A non-transitory computer-readable medium storing instructions thereon that, when executed by a processing unit (102) of a wearable electronic device, cause the wearable electronic device to perform a method comprising the steps of:
a. collecting data from a multi-modal sensor array (500);
b. processing the data using a synergistic fusion algorithm; and generating a composite health metric based on the processed data.

20. The non-transitory computer-readable medium of claim 19, wherein the composite health metric is a cuffless blood pressure estimate.
